(19) United States
(12) Patent Application Publication
(10) Pub. No.: US 2008/0306211 A1
(43) Pub. Date: Dec. 11, 2008

(54) Process for prepapring polycerol and mixed ethers

(75) Inventors: Daniel W. Lemke, Cokato, MN (US); Scott Nivens, Minneapolis, MN (US)

Correspondence Address: CARGILL, INCORPORATED, LAW/24, 15407 MCGINTY ROAD WEST, WAYZATA, MN, 55391, US

(73) Assignee: CARGILL, INCORPORATED, Wayzata, MN (US)

(21) Appl. No.: 12/221,608

(22) Filed: Aug. 4, 2008

Related U.S. Application Data
(63) Continuation of application No. PCT/US2007/003085, filed on Feb. 6, 2007.

(51) Int. Cl. C08G 65/34; C07C 41/09

(52) U.S. Cl. 525/50; 528/425; 568/619

(57) ABSTRACT

Disclosed is a method of reacting glycerol to form polyglycerol or mixed ethers of glycerol or other alcohols (e.g., polyols). In some embodiments, the method comprises reacting glycerol in the presence of a fatty acid-containing compound and a metal catalyst to form polyglycerol. In other embodiments, the method comprises reacting glycerol and/or an alcohol in the presence of a fatty acid-containing compound and a metal catalyst to form a mixed ether.

### CROSS-REFERENCE TO RELATED APPLICATIONS

This application claims the benefit of U.S. Provisional Application Ser. No. 60/765,843, filed Feb. 6, 2006, the disclosure of which is incorporated herein by reference.

### FIELD

The present invention relates to an improved method of making polyglycerol and mixed ethers of polyols such as glycerol.

### BACKGROUND

Polyglycerol manufacturers have generally focused on one of two paths in the synthesis of polyglycerol from glycerol. In a first path, glycerol is directly polymerized using a condensation catalyst. This process is described, for example, in U.S. Pat. No. 3,637,774. In a second path, polymerization of glycerol, diglycerol, or polyglycerol is accomplished with the use of epichlorohydrin. This process is described, for example, in U.S. Pat. No. 4,960,953.

Although the epichlorohydrin process is capable of producing high quality polyglycerol, the process has several drawbacks. For example, the process requires a high capital investment in equipment since it must be conducted in an explosion-proof facility that is capable of handling epichlorohydrin, which is flammable and a carcinogen. Post-reaction processing is also extensive, typically including, for example, solid-liquid extraction to remove the crystalline salt byproduct, followed by ion exchange, dehydration, and short path distillation. The epichlorohydrin process also requires significant process control, including close control and monitoring of the addition of the epichlorohydrin in order to control the heat of the reaction.

The direct process also suffers from several drawbacks. In the direct process, the rate of reaction is controlled, at least in part, by the ability of the process equipment to effectively remove the water that is formed during the reaction (i.e., “water of reaction”). In order to accomplish this, the process equipment typically includes a large multi-plate packed distillation column with recycle, which functions to separate the water of reaction as a distillate from the reacting glycerol and polyglycerol. In order to control the removal of the water of reaction, both the temperature and the level of vacuum in the process must be closely controlled and adjusted as the reaction proceeds. During a standard ether condensation process, the process control operator is continuously adjusting the equipment in order to keep just enough glycerol refluxing in the packed column in order to drive the removal of the water of reaction, while at the same time preventing a sudden batch foam over. This problem is especially prevalent when the rate of generation of water of reaction is high, for example, at the beginning of the reaction.

In view of the foregoing, what is desired is an industrially convenient route to the production of polyglycerol and mixed ethers of polyols.

### SUMMARY

The invention provides a method of reacting glycerol to form polyglycerol or mixed ethers of glycerol or other alcohols (e.g., polyols). In some embodiments, the method comprises reacting glycerol in the presence of a fatty acid-containing compound and a metal catalyst to form polyglycerol. In other embodiments, the method comprises reacting glycerol and/or an alcohol in the presence of a fatty acid-containing compound and a metal catalyst to form a mixed ether.

Advantageously, the addition of the fatty acid-containing compound allows the reduction of the metal catalyst to a level of about one third or less of the level that is typically used in glycerol condensation reactions, without negatively affecting the rate of the reaction. In addition, the presence of the fatty acid-containing compound acts as an effective in-process defoaming agent; in certain implementations, this can provide enough lipophillic character to the reaction mixture that the water of reaction may be removed without having to reflux the glycerol. Accordingly, some methods of the invention can greatly simplify the manufacture of polyglycerol and ethers of glycerol, essentially allowing standard etherification or esterification equipment to be used, and without the need for a large packed distillation column, associated distillation and recycle equipment, and complex in-process computer control.

### BRIEF DESCRIPTION OF THE FIGURES

FIG. 1 is a representative reaction scheme for the production of polyglycerol.

FIG. 2 is representative reaction scheme for the production of a mixed ether of glycerol using pentaerythritol.

FIG. 3 is a representative reaction scheme for the production of a mixed ether of glycerol using a fatty alcohol.

FIG. 4 is a chromatograph trace of polyglycerol.

FIG. 5 is a chromatograph trace of the mixed ether of glycerol of Example 4.

FIG. 6 is a graph showing certain in-process data for the reaction of Example 4.

### DETAILED DESCRIPTION

The invention provides an improved method of making polyglycerol or mixed polyol ethers, such as mixed polyol ethers of glycerol or other alcohols (e.g., polyols).

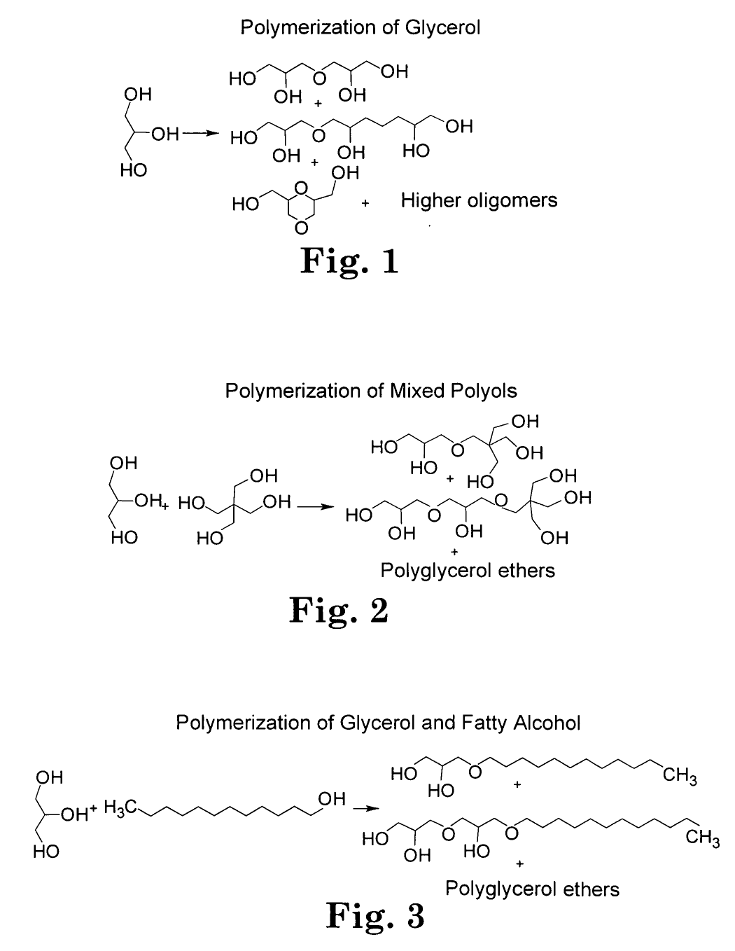

In some embodiments, the method comprises polymerizing glycerol in the presence of a fatty acid-containing material and a metal catalyst to form polyglycerol. More specifically, one such method comprises combining: (i) glycerol (typically about 20-99 % wt., more preferably about 75-98% wt.); (ii) a fatty acid-containing material (e.g., fatty acid, fatty ester, polycarboxylic acid, or triglyceride) (typically about 0.2-20% wt., more preferably about 0.5-5% wt.); and (iii) a metal catalyst (e.g., an alkali metal catalyst) (typically about 0.1-2% wt., more preferably about 0.2-0.5% wt.) in a reaction vessel and heating the reactants to a temperature sufficient to cause the glycerol to react to form polyglycerol. For example, the reactants are typically heated to a temperature of about 200° C. to about 250° C. In many embodiments, the reaction is conducted under an inert atmosphere, such as under a sparge of nitrogen, and at a reduced pressure, for example, from about 140 mmHg to about 300 mmHg. A representative reaction scheme for the production of polyglycerol is shown in FIG. 1.

In some embodiments, the method comprises forming a mixed ether of a polyol (e.g., glycerol) by reacting a polyol and an alcohol in the presence of a fatty acid-containing material and a metal catalyst. More specifically, the method comprises combining: (i) a polyol (e.g., glycerol) (typically about 20-99% wt., more preferably about 75-98% wt.); (ii) an alcohol (e.g., fatty alcohol or polyol) (typically about 1-80% wt., more preferably 10-30 % wt.); (iii) a fatty acid-containing material (e.g., fatty acid, fatty ester, poly-carboxylic acid, or triglyceride) (typically about 0.2-20% wt., more preferably about 0.5-5% wt.); and (iii) a metal catalyst (e.g., an alkali metal catalyst) (typically about 0. 1-2% wt., more preferably about 0.2-0.5% wt.) in a reaction vessel and heating the materials to a temperature sufficient to cause the glycerol and alcohol to react to form the mixed ether of glycerol. Representative reaction schemes are shown in FIGS. 2-3. In FIG. 2 pentaerythritol is shown being reacted with glycerol to form a mixed ether of glycerol. In FIG. 3, a fatty alcohol is shown being reacted with glycerol to form a mixed ether of glycerol.

The methods of the invention make use of a metal catalyst to promote the reaction of glycerol or other polyol. In many embodiments, the metal catalyst comprises a salt (e.g., a hydroxide) of a group IA metal (i.e., an alkali metal) or a salt of a group IIA metal (i.e., an alkaline earth metal). Representative examples of metal catalysts include sodium metal, potassium metal, potassium hydroxide (KOH), sodium hydroxide (NaOH), calcium hydroxide (Ca(OH₂)), tin oxylate, and the like. Also useful are carbonate, acetate, phosphate, sulfate, alkoxylate, or oxide salts of the metals. Mixtures may also be used. In exemplary embodiments, the metal catalyst is potassium hydroxide (KOH).

Although other amounts may be useful, the metal catalyst is typically added in an amount ranging from about 0.1% wt. to about 2% wt., more typically ranging from about 0.2% wt. to about 0.5% wt. of the total weight of the reactants.

The methods of the invention are conducted in the presence of a minor amount of a fatty acid-containing compound. In many embodiments, the fatty acid-containing compound functions to reduce the amount of metal catalyst that is needed to conduct the reaction and additionally acts as an in-process defoaming agent. The presence of the fatty acid-containing compound allows the reaction to be conducted using a conventional etherification or esterification apparatus, which typically includes a stainless steel or glass-lined kettle equipped with heating, agitation, an over heat condenser, a receiver, an inert gas (sparge and purge) and a vacuum control). That is, the reaction can be conducted without the need for a large packed distillation column, associated distillation and recycle equipment, and advanced in-process computer control equipment.

Examples of useful fatty acid-containing compounds include fatty acids, fatty esters, and mixtures thereof. Fatty esters include both fatty esters of monofunctional alcohols (e.g., fatty esters of methanol, ethanol, and the like) and fatty esters of polyfunctional alcohols (e.g., fatty esters of glycerol, sorbitol, and the like). Fatty esters of glycerol include, for example, glycerides, such as monoglycerides, diglycerides, triglycerides, and mixtures thereof.

Typically, the acyl portion of the fatty acid or acyl portion of the fatty ester has a carbon chain length ranging from about 2 to about 30 carbon atoms, more typically about 8 to about 18 carbon atoms. The acyl portion may be saturated, unsaturated, branched, cyclic, or substituted.

Representative examples of fatty acids include lauric acid, stearic acid, isostearic acid, oleic acid, palmitic acid, behenic acid, myristic acid, caprylic acid, capric acid, caproic acid, arachidic acid, myristoleic acid, linoleic acid, linolenic acid, licaneic acid, ricinoleic acid, eleostearic acid, and erucic acid. Fatty esters that contain any of these fatty acids may also be suitable.

When a glyceride (e.g., a triglyceride) is used, the glyceride may be hydrogenated, non-hydrogenated, fractionated, or may be a combination of different glycerides. Representative examples of triglyceride oils include soybean oil, coconut oil, canola oil, corn oil, palm oil, linseed oil, tallow, lard, sunflower oil, and the like. Fractions and hydrogenated versions of the triglyceride oils may also be used. Exemplary triglyceride oils include alkali refined sunflower and coconut oils.

Also useful are diacid compounds, for example, having a carbon chain length ranging from about 2 to about 40 carbon atoms. Representative examples include adipic acid, oxalic acid, malonic acid, succinic acid, gluconic acid, pimelic, phthalic acid, sebacic acid, azelaic acid, trimellitic acid, and dimer fatty acids. Acid anhydrides may also be used.

Advantageously, in some embodiments, the use of a diacid compound (e.g., adipic acid) results in the formation of a product that is stable as a homogeneous single phase. This allows and end-user to use the product without mixing prior to use. In addition, in some embodiments, the use of diacids, which do not form monoglycerides, results in improved processing since monoglycerides tend to distill which may cause the condenser to become fouled or plug. In some embodiments, the use of Ca(OH)₂ metal catalyst along with a diacid (e.g., adipic acid) results in the formation of a product that is lighter in color and is substantially free of haze.

Although other amounts may be useful, in many embodiments, the methods of the invention are conducted in the presence of a minor amount of a fatty acid-containing compound, for example, about 50% wt. or less, about 40% wt. or less, about 30% wt. or less, or about 20% wt. or less of the reactive mixture. For example, in some embodiments, the fatty acid-containing composition is present in an amount ranging from about 0.2% wt. to about 20% wt. of the reactive mixture. In more typical embodiments, the fatty acid-containing composition is present in an amount ranging from about 0.5% wt. to about 5 % wt. of the reactive mixture.

In some embodiments, a second alcohol is included in the reactive mixture along with glycerol. The addition of a second alcohol results in the formation of mixed ethers of glycerol. Useful alcohols may be monofunctional alcohols (e.g., a fatty alcohol) or polyfunctional alcohols (i.e., polyols). Representative examples of alcohols include trimethylol propane, pentaerythritol, manitol, stearyl alcohol, sorbitol, ricinoleic acid, polyethylene glycol, polypropylene glycol, butane diol, hexane diol, and the like.

In many embodiments, the reaction is conducted at a temperature ranging from about 200° C. to about 250° C., more typically ranging from about 210° C. to about 240° C. Typically, the reaction is conducted under a reduced pressure, for example, ranging from about 140 mmHg to about 300 mmHg. The preferred reaction pressure typically ranges from about 10 mmHg to about 60 mmHg greater than the vapor pressure of glycerol at the temperature of the reaction. That is, if the vapor pressure of glycerol is about 145 mmHg at 230° C., then a preferred range for the reaction pressure would be about 155 mmHg to about 205 mmHg.

In a typical reaction, the reactive composition is heated to the target reaction temperature (e.g., about 230° C.). At a temperature of about 100° C., vacuum is applied and the level of vacuum is set according to the desired batch temperature as described above. For example, when the batch temperature reaches about 100° C. to about 160° C., the pressure is about 400 mmHg; when the batch temperature reaches about 160° C. to about 190° C., the pressure is about 300 mmHg; when the batch temperature reaches about 190° C. to about 210° C., the pressure is about 250 mmHg; and when the batch temperature reaches about 210° C. to about 230° C., the pressure is about 160 to 200 mmHg.

The extent of reaction may be monitored, for example, by hydroxyl value, refractive index, viscosity, gas chromatography, or other suitable technique. In some embodiments, the reaction is considered to be complete, as measured by gas chromatography, when the area count of free glycerol is between about 20% to about 40% of total area count of the gas chromatograph trace. If hydroxyl value is used, a hydroxyl value of about 1250 or less is typically considered to be complete reaction.

Upon completion, free (i.e., unreacted) glycerol may be left in the product or may be distilled out, for example, to a level of about 3% wt. or less in the final product. To remove unreacted glycerol, the reaction kettle is typically cooled to about 210° C. or less and a vacuum of about 10 mmHg or less is applied. Steam stripping may also be used to remove unreacted glycerol. Gas chromatography or other analytical technique may be used to monitor the stripping process. When the target level of unreacted glycerol is reached, the mixture is typically cooled under vacuum to a temperature of about 80° C. The final product can then filtered, for example, by passing it through a 10-micron sock at about 60° C. to about 80° C.

In many embodiments, polyglycerol formed according to the method of the invention comprises about 75% wt. or more of diglycerol, triglycerol, and tetraglycerol species, and comprises less than about 10% wt. of polyglycerol species of hexaglycerol and greater. Advantageously, this distribution is in accordance with the requirements set by the European and Japanese governments for polyglycerols that are used in the food and cosmetic products. (see, e.g., CAS no. 25618-55-7). In some embodiments, the polyglycerol is polymerized to a higher degree of conversion than described above. Typically, as the glycerol level is depleted, the level of cyclic polyglycerol that is formed increases. In some embodiments, the cyclic polyglycerol ranges from about 5% wt. to about 90% wt.

The presence of the fatty acid-containing compound in the reaction mixture typically results in the formation of ester functional groups in some of the polyglycerol compounds prepared in accordance with the method of the invention. If an ester-free product is desired, the polyglycerol may be distilled in order to separate the pure polyol fraction from the ester-containing fraction. However, in many instances, the polyglycerols comprising at least some ester-containing species are acceptable in view of the fact that many industrially important end-uses for polyglycerol include the subsequent conversion of the polyglycerol into an ester.

In some embodiments, the presence of the fatty acid-containing compound causes the final polyglycerol product to exhibit some phase separation upon setting. Typically, the phase separation can be reversed with slight mixing to yield a homogeneous mixture. Alternatively, the addition of small amount of water (e.g., about 10% wt. or less water) typically provides a clear and phase stable mixture.

Advantageously, in some embodiments, the fatty acid-containing compound allows the reduction of the metal catalyst to a level of about one third or less of the level that is typically used in glycerol condensation reactions, without negatively affecting the rate of the reaction. The presence of the fatty acid-containing compound also acts as an effective in-process defoaming agent; in certain implementations, this can provide enough lipophillic character to the reaction mixture that the water of reaction may be removed without having to reflux the glycerol. Accordingly, some methods of the invention can greatly simplify the manufacture of polyglycerol and ethers of glycerol, essentially allowing standard etherification or esterification equipment (e.g., stainless steel or glass lined kettle equipped with heating, agitation, over heat condenser, receiver, inert gas, and vacuum control) to be used. That is, some embodiments of the invention may be conducted without the need for a large packed distillation column, associated distillation and recycle equipment, and complex in-process computer control equipment. In some embodiments, it is desirable to include an in-line demister or cyclone in order to minimize glycerol loss due to entrapment.

The invention will now be further described with reference to the following non-limiting examples.

### EXAMPLES

### Analytical Test Procedures

1. GC method for polyglycerol determination: A 10-50 mg sample of polyglycerol was derivatized with 1 ml of silylating reagent (hexamethyidisilazane (HMDS): trimethylchlorosilane (TMCS): pyridine, 3:1:9 from Supelco, Inc. of Bellefonte, Pa.) following Supelco's recommended procedure.

The polyglycerol distribution was determined by gas chromatography using a DB-5HT capillary column (30 m×0.32 mm ID×0.1 mm film thickness), available from J & W Scientific Inc. of Folsom, Calif., and an HP Series 5890 Chromatograph equipped with an FID detector. The splitter was set at 20:1 and the FID set with flow rates of 30 ml/min hydrogen; 30 ml/min purge gas; and 300 ml/min air.

The conditions for the column were: helium carrier gas (6-8 lbs column head pressure); injector temperature (375° C.); detector temperature (375° C.); temperature program (70-375° C. at 10° C./min ramp, 5 min at 100° C. and 5 min at 375° C.); and a 1 μl injection volume. To avoid overloading the column, attempts were made to keep the total area count within the range of 3-8,000,000.

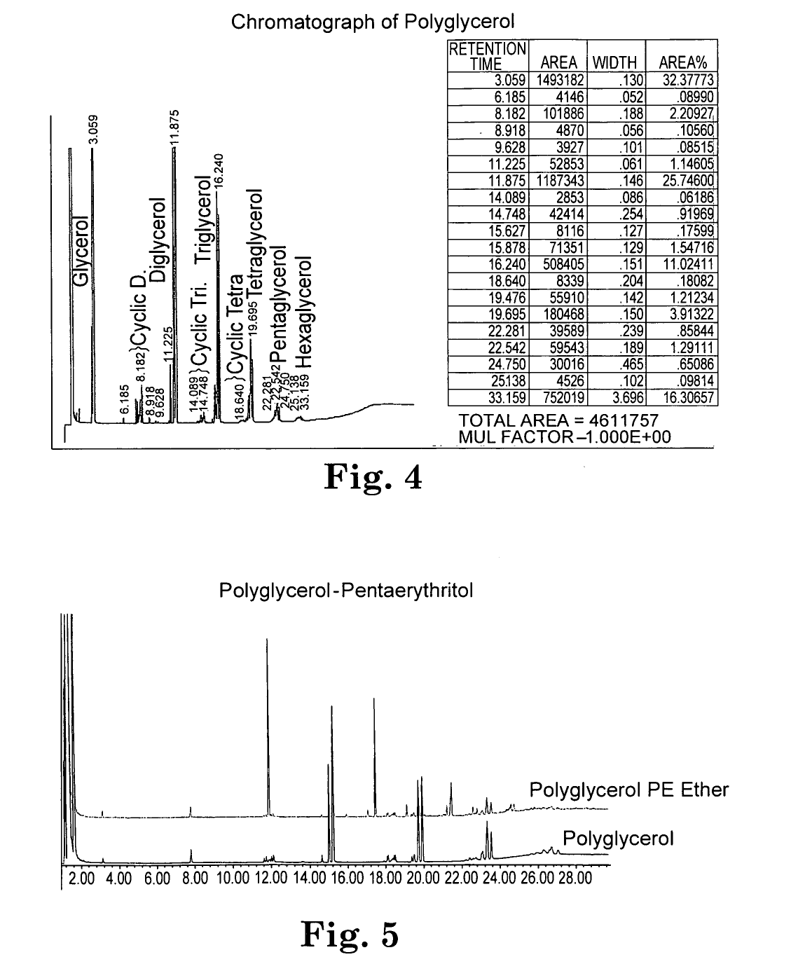

The peaks were identified by GCMS. A typical chromatograph is shown in FIG. 4.

2. Analysis of Unknown Components by Gas Chromatography coupled to Mass Spectrometry (GC/MS): Each of the preparations was also analyzed by GC/MS to identify any unknown compounds present in the samples. The polyglycerol preparations were chromatographed on a non-polar stationary phase (DB5-HT, 30m×0.25 mm×0.20 mm). The temperature program was 100° C. (5 minutes) to 295° C. at 10° C./min (10 minutes). Helium was the carrier gas and the inlet pressure was 48.2 psi in the constant flow mode at 1.5 mL/min. The split ratio was approximately 10:1. The injector and detector temperatures were 325° C. The mass spectra were collected using an Agilent quadrapole mass spectrometer with source temperature of 230° C. and voltage of 70 eV. A full scan was performed from 15 to 700 Daltons.
3. Hydroxyl Value: Hydoxyl value was measured in accordance with AOCS Cd 13-60.
4. Acid Value: Acid value was measure in accordance with AOCS Te 2a-64 (97).
5. Ash: Ash was measured in accordance with Ca 11-55 (03) using a 2 gram sample.
6. Moisture (ppm): Moisture was measured in accordance with AOCS Ca 2e-84 (97).
7. Color (Gardner): Gardner color was measure in accordance with AOCS Td 1a-64 (00).
8. Kinematic Viscosity: Kinematic viscosity was measure in accordance with ASTM D-445.
9. Density: Density was measure in accordance with ASTM D1475.

### Example 1

### Preparation of Polyglycerol Using Potassium Hydroxide

Into a two liter four neck flask was placed USP glycerol (99.7%, 1,195 g, 99.6% wt.) and potassium hydroxide pellets (4.8 g, 0.4% wt.). The flask was fitted with an agitator, nitrogen sparge, horizontal condenser connected to a four inch vertical offset, 500 mL receiver and temperature and vacuum control. Nitrogen sparge was set at a medium flow; the agitator was turned on and the mixture heated to 230° C. at a rate of 1.5° C./min. At 230° C., the vacuum was ramped to 160 mmHg over a one-hour period. When the vacuum of 160 mmHg was achieved, the batch was sampled periodically and analyzed by GC. After 24 hours at 230° C. the reaction was stopped because the Hydroxyl Value did not drop below 1500 and the GC showed that 56% glycerol remained.

### Example 2

### Preparation of Polyglycerol Using High Oleic Sunflower Oil, Potassium Hydroxide, Fresh Glycerol and Recovered Glycerol

The reaction vessel used consisted of a 10,000 gallon stainless steal reactor equipped with a vertical twelve inch diameter fifteen foot packing filled demister column with an overhead condenser, nitrogen sparge-purge, receiver and four stage jet vacuum system. Careful monitoring of the demister column showed that it was not capable of fractionating condensation codistillation mixtures (i.e., glycerol water mixtures). When no attention was given to the column the vapor temperature readily rose to within a few degrees of the kettle temperature signifying that the vapor was an aspirated kettle mixture and not a codistillation product of glycerol and water. When the column condenser temperature was set at 50° C. the returning vapor condensate was more than enough to limit the glycerol carry over. At the end of the reaction the receiver showed the water of reaction to contain no more than 1% glycerol. When the column temperature was not controlled the water of reaction contained 10% wt. glycerol. The polymerization reaction was monitored by GC. Two preparations were made. In Prep 1, the polymerization was stopped at 20% residual glycerol and then the free glycerol was stripped to less than 3% wt. In Prep 2, the recovered glycerol was recycled back into the polymerization process.

### Prep 1

The nitrogen sparge was set at 2 cfm. The reactor was charged with USP glycerol (99.7%, 30,280 kg, 96.6% wt.), high oleic sunflower oil (928 kg, 3.0% wt.) and potassium hydroxide pellets (124 kg, 0.4% wt.). The mixture was heated to 230° C. The condenser cooling water was set at 75° C. When the temperature reached 120° C. the vacuum was ramped to 400 mmHg. When the temperature reached 230° C., the vacuum was ramped to 160 mmHg over six hours. After ten hours at 160 mmHg, the batch was sampled and was thereafter sampled every two hours. The in-process data is shown in the TABLE 2-1.

TABLE 2-1

| Time (hrs:min) | ° C. | mmHg | Remarks |
|---|---|---|---|
| 0 | 20 | 685 | Charge, nitrogen at 2 cfm, heat to 230° C., ramp to 400 mmHg, |
| | | | condenser cooling water temperature set to 75° C. |
| 3 | 126 | 400 | |
| 4:30 | 230 | 400 | Ramp to 160 mmHg over six hours |
| 8:40 | 230 | 200 | Three foot from condenser vapor temperature was 216° C. and |
| | | | rising. Reduced cooling temp to 50° over two hours |
| 14:10 (0:00) | 230 | 160 | At target vacuum, hold |
| 16:00 (1:50) | 230 | 160 | Receiver density 1.10 g/ml |
| 18:00 (3:50) | 230 | 160 | Receiver density 1.03 g/ml |
| 22:10 (8:00) | 230 | 160 | 29% wt. glycerol, three foot from condenser vapor temperature was |
| | | | 198° C. |
| 24:10 (10:00) | 230 | 160 | 25.8% glycerol, raise pressure to slow reaction, |
| 25:50 (11:40) | 231 | 280 | Difficult to slow distillate from coming over but finally stopped at |
| | | | 300 mmHg. |
| 26:10 (12:00) | 233 | 300 | 19.2% wt. glycerol, receiver density 1.00 g/ml, set up to strip, cool |
| | | | to 205° C. |
| 28:10 (14:00) | 205 | 300 | Apply full vacuum |
| 29:10 (15:00) | 205 | 29 | |
| 33:10 (19:00) | 203 | 10 | 8.3% wt. glycerol, distilling |
| 35:45 (21:35) | 206 | 7 | 5.2% wt. glycerol |
| 37:50 (23:40) | 205 | 6 | 2.9% wt. glycerol, cool under vacuum |
| 42:15 (28:05) | 91 | 6 | Break vacuum with nitrogen. Water was added at 5.0% wt. to cut |
| | | | viscosity and improve handling. Recovered 6000 of glycerol from |
| | | | receiver. |

The resulting polyglcerol was analyzed and the properties are shown in TABLES 2-2 and 2-3.

TABLE 2-2

| Component | % Wt. |
|---|---|
| Glycerol | 3.3 |
| Diglycerol | 27.7 |
| Triglycerol | 21.4 |
| Tetraglycerol | 16.9 |
| Pentaglycerol | 10.8 |
| Hexaglycerol | 8.3 |
| Heptaglycerol | 5.6 |
| Octaglycerol | 4.0 |
| Cyclic diglycerol | 2.1 |
| Cyclic triglycerol | ND |
| Cyclic tetraglycerol | ND |

TABLE 2-3

| | |
|---|---|
| OH Number | 980 |
| AV | 0.4 |
| Color | 3 |
| Moisture | 4.8 |
| Appearance | Haze |
| Density | 1.26726 |
| Viscosity (60° C., cP) | 4000 |
| Odor | Mild |

Prep 2

For this reaction the nitrogen sparge was set at 2 cfm. The reactor was charged with USP glycerol (99.7%, 21,970 kg, 75.9% wt.), recycled glycerol (6,000 kg, 20.7% wt., from Prep 1), high oleic sunflower oil (869 kg, 3.0% wt.) and potassium hydroxide pellets (116 kg, 0.4% wt.). The mixture was heated to 230° C. The condenser cooling water was set at 50° C. When the temperature reached 120° C. the vacuum was ramped to 400 mmHg. At 230° C., the vacuum was ramped to 160 mmHg over six hours. After ten hours at 160 mmHg, the batch was sampled, and was then sampled every two hours thereafter. The in-process data is shown in TABLE 2-4.

TABLE 2-4

| Time (hrs:min) | ° C. | mmHg | Remarks |
|---|---|---|---|
| 0 | 20 | 680 | Charge, nitrogen at 2 cfm, heat to 120° C., ramp to 400 mmHg, |
| | | | condenser cooling water temperature set to50° C. |
| 0:50 | 120 | 400 | At 120° C., heat to 230° C. |
| 6:10 | 230 | 400 | At target temperature, water coming over at a rate of 2 kg/min, three |
| | | | foot from condenser vapor temperature was 87° C., ramp to 160 mmHg |
| | | | over six hours |
| 7:50 | 229 | 300 | Three foot from condenser vapor temperature was 81° C. and rising. |
| 9:35 | 231 | 200 | Three foot from condenser vapor temperature was 112° C. and rising. |
| 10:15 | 229 | 160 | Receiver density 1.025 |
| 14:55 (0:00) | 230 | 160 | Receiver density 1.008 Three foot from condenser vapor temperature |
| | | | was 165° C. and holding. |
| 23:15 (8:20) | 230 | 157 | Glycerol at 42.1% wt. Three foot from condenser vapor temperature |
| | | | was 167° C. and holding. |
| 24:15 (9:20) | 229 | 154 | Glycerol at 36.1% wt. Three foot from condenser vapor temperature |
| | | | was 117° C. and dropping. |
| 25:15 (10:20) | 228 | 165 | Three foot from condenser vapor temperature was 100° C. and |
| | | | dropping. |
| 26:05 (11:10) | 200 | 300 | Receiver density 1.0029. Cooled batch, reset vacuum to 160 mmHg, |
| 27:40 (12:45) | 90 | 160 | Break vacuum with nitrogen, add 1500 kg of water to reduce |
| | | | viscosity. |

The resulting polyglcerol was analyzed and the properties are shown in TABLES 2-5 and 2-6.

TABLE 2-5

| Component | % Wt. |
|---|---|
| Glycerol | 36.3 |
| Diglycerol | 27.8 |
| Triglycerol | 15.1 |
| Tetraglycerol | 6.3 |
| Pentaglycerol | 3.8 |
| Hexaglycerol | 1.8 |
| Heptaglycerol | 0 |
| Octaglycerol | 0 |
| Cyclic diglycerol | 4.5 |
| Cyclic triglycerol | 2.3 |
| Cyclic tetraglycerol | 2.2 |

TABLE 2-6

| | |
|---|---|
| OH Number | 1350 |
| AV | 0.2 |
| Color | 2 |
| Moisture | 5.0 |
| Appearance | Clear |
| Density | 1.24569 |
| Viscosity (60° C., cP) | 4600 |
| Odor | Mild |

### Example 3

### Preparation of Polyglycerol Using Adipic Acid, Potassium Hydroxide, and Fresh Glycerol

Into a two liter four neck flask fitted with nitrogen sparge, condenser, receiver and vacuum capabilities was placed glycerol (99.7%, 1529 g, 99.2% wt.); adipic acid (7.65 g, 0.50% wt.); and potassium hydroxide pellets (4.56 g, 0.30% wt.). Nitrogen sparge was set at a medium flow. The mixture was heated to 230° C. at a rate of 1.5° C./min. At 230° C., the vacuum was ramped to 160 mmHg. When the vacuum of 160 mmHg was achieved, the batch was sampled periodically by GC. The reaction proceeded as follows: 0 hrs (98.8% glycerol); 8.6 hrs (63.8% glycerol); 17.3 hrs (43.3% glycerol); and 22.9 hrs (32.2% glycerol). The final composition was as follows: glycerol (32.2%); cyclic diglycerol (2.9%); diglycerol (29.4%); cyclic triglycerol (1.6%); triglycerol (17.6%); cyclic tetraglycerol (0.2%); tetraglycerol (7.5%); pentaglycerol (4.7%); hexaglycerol (0.9%); heptaglycerol and higher (<1%).

### Example 4

Preparation of Mixed Polyglycerol Polyol (pentaerythritol)

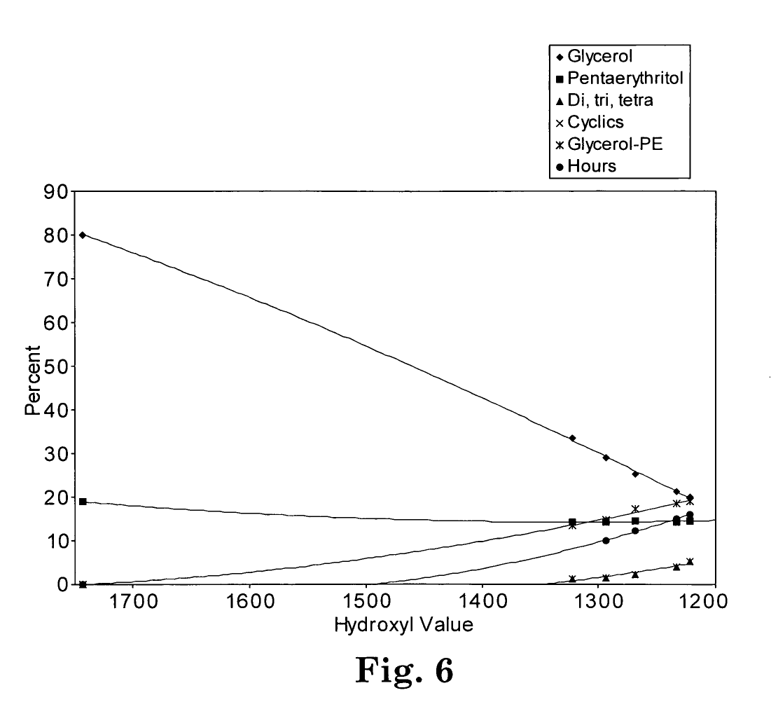

Into a 50 gallon pilot reactor was placed USP glycerol (99.7%, 410 lbs., 77.3% wt.), alkali refined deodorized sunflower oil (16.2 lbs., 3.1% wt.), technical grade pentaerythritol (230, 19.2 % wt.) and potassium hydroxide pellets (2.2 lbs. 0.4% wt.). The same equipment set up and procedure as seen in Example I was followed. The reaction was stopped after 16 hours at 230° C. The composition of the reaction mixture and Hydroxyl Values can be seen in FIG. 6. The reaction temperature was decreased to 208° C. and the vacuum increased to 5 mmHg. After 7 hours, the composition was as follows: glycerol (2.4%); cyclic diglycerol (2%); diglycerol (22.1%); cyclic triglycerol (<0.2%); triglycerol (16.1%); cyclic tetraglycerol (<0.1%); tetraglycerol ( 6.3%); pentaglycerol ( 4.9%); hexaglycerol (1.1%); heptaglycerol and higher (<1%); pentaerythritol (17.7%); glycerol-pentaerythritol (13.1%); two glycerol-pentaerythritol (9.1%); three glycerol-pentaerythritol (4.0%); four glycerol-pentaerythritol (0.8%); and five glycerol-pentaerythritol (0.4%). The final analysis showed a Hydroxyl Value of 1050, viscosity 3320 cP @60° C. and a 2+ Gardner in color. FIG. 5 is a chromatograph of the product of Example 4 with a chromatograph of polyglycerol over-laid to assist in distinguishing the glycerol-pentaerythritol peaks from the polyglycerol peaks. 130.9 lbs of glycerol was recovered, which was 24.5% wt. of the total charge. Note: the vacuum was reduced to as low as 121 mmHg and held below 150 mmHg in an effort to determine the limits of the process. As a result, excessive glycerol was lost to the receiver during the reaction.

### Example 5

### Preparation of Mixed Polyglycerol Fatty Alcohol

Charge USP glycerol (99.7%, 928 g, 77.3% wt.), alkali refined deodorized sunflower oil (37 g, 3.1% wt.), oleyl alcohol (230, 19.2% wt.) and potassium hydroxide pellets (4.8 g, 0.4% wt.) in a two-liter, four-neck flask. Following the same equipment set up and procedure as seen in Example 1, stop the reaction after 16 hours at 230° C. The water of reaction will contain about 10% wt. glycerol. The product will have a Hydroxyl Value of about 917, a viscosity of about 1100 cP @ 60° C. and about 1 Gardner in color. Decrease the reaction temperature to about 208° C. and increase the vacuum to about 5 mmHg. After 7 hours the composition will be generally as follows: glycerol (2.4%); cyclic diglycerol (2%); diglycerol (22.1%); cyclic triglycerol (<0.2%); triglycerol (16.1%); cyclic tetraglycerol (<0.1%); tetraglycerol ( 6.3%); pentaglycerol (4.9%); hexaglycerol (1.1%); heptaglycerol and higher (<1%); pentaerythritol (17.7%); glycerol-oley alcohol (13.1%); two glycerol-oley alcohol (9.1%); three glycerol-oley alcohol (4.0%); four glycerol-oley alcohol (0.8%); and five glycerol-oley alcohol (0.4%). The final analysis shows a Hydroxyl Value of about 732, viscosity of about 2200 cP @60° C. and 2+ Gardner in color. About 220 g of glycerol, 18.2% wt. of total charge, will be recovered.

Other embodiments of this invention will be apparent to those skilled in the art upon consideration of this specification or from practice of the invention disclosed herein. Variations on the embodiments described herein will become apparent to those of skill in the relevant arts upon reading this description. The inventors expect those of skill to use such variations as appropriate, and intend to the invention to be practiced otherwise than specifically described herein. Accordingly, the invention includes all modifications and equivalents of the subject matter recited in the claims as permitted by applicable law. All patents, patent documents, and publications cited herein are hereby incorporated by reference as if individually incorporated. In case of conflict, the present specification, including definitions, will control.

1-64. (canceled)

65. A method of making polyglycerol comprising: reacting a reactive mixture comprising a fatty acid-containing compound; a metal catalyst; and at least about 20 weight percent (% wt.) glycerol to form polyglycerol.

66. The method of claim 1, wherein the fatty acid-containing compound is present in an amount ranging from about 0.2% wt. to about 20% wt. of the reactive mixture.

67. The method of claim 1, wherein the fatty acid-containing compound is present in an amount ranging from about 0.5% wt. to about 5% wt. of the reactive mixture.

68. The method of claim 1, wherein the glycerol is present in an amount of at least about 75% wt. of the reactive mixture.

69. The method of claim 1, wherein the glycerol is present in an amount ranging from about 75% wt. to about 98% wt. of the reactive mixture.

70. The method of claim 1, wherein the fatty acid-containing compound comprises a diacid.

71. The method of claim 70, wherein the diacid has a carbon chain length ranging from about 2 to about 40 carbon atoms.

72. The method of claim 70, wherein the diacid is selected from adipic acid, oxalic acid, malonic acid, succinic acid, gluconic acid, pimelic, phthalic acid, sebacic acid, azelaic acid, trimellitic acid, dimer fatty acids, mixtures thereof, and acid anhydrides thereof.

73. The method of claim 70, wherein the polyglycerol is a homogeneous single phase.

74. The method of claim 70, wherein the acid is adipic acid and the metal catalyst is Ca(OH)₂.

75. The method of claim 1, wherein the metal catalyst is an alkali metal catalyst or an alkaline earth metal catalyst.

76. The method of claim 1, wherein the metal catalyst is present in an amount ranging from about 0.1% wt. to about 2% wt.

77. The method of claim 1, wherein the metal catalyst is present in an amount ranging from about 0.2% wt. to about 0.5% wt.

78. The method of claim 1, wherein the polyglycerol comprises about 75% wt. or greater of diglycerol, triglycerol, and tetraglycerol species; and about 10% wt. or less of polyglycerol species of hexaglycerol and greater.

79. The method of claim 1, wherein the polyglycerol comprises a cyclic content of about 5% wt. to about 90% wt.

80. The method of claim 1, wherein the polyglycerol is manufactured in an apparatus that does not include a distillation column.

81. The method of claim 1, wherein the polyglycerol is manufactured in an etherification or esterification apparatus.

82. Polyglycerol produced according to the method of claim 1.

83. A method of making a mixed ether of glycerol comprising: reacting a reactive mixture comprising glycerol; an alcohol; a fatty acid-containing compound; and a metal catalyst to form the mixed ether of glycerol, wherein the glycerol is present in an amount of at least about 20 weight percent (% wt.) in the reactive mixture and the alcohol is present in an amount of at least about 10% wt. in the reactive mixture.

84. The method of claim 83, wherein the alcohol comprises a monofunctional alcohol or a polyol.

85. The method of claim 83, wherein the alcohol is selected from the group consisting of trimethylol propane, pentaerythritol, manitol, stearyl alcohol, sorbitol, ricinoleic acid, polyethylene glycol, polypropylene glycol, butane diol, hexanel diol, and mixtures thereof.

86. The method of claim 83, wherein the alcohol is present in an amount ranging from about 10% wt. to about 30% wt. of the reactive mixture.

87. A mixed ether of glycerol prepared according to the method of claim 83.

88. A method of making polyglycerol comprising: adding to a reaction vessel a reactive mixture comprising:
(i) about 20% wt. to about 99% wt. glycerol;
(ii) about 20% wt. or less a fatty acid-containing compound; and
(iii) a metal catalyst; and
reacting the reactive mixture to form polyglycerol.

89. A method of making a mixed ether comprising: reacting a reactive mixture comprising an alcohol; a fatty acid-containing compound; and a metal catalyst to form the mixed ether.
